(19) United States
(12) Patent Application Publication
(10) Pub. No.: US 2026/0345519 A1
(43) Pub. Date: Nov. 5, 2026

(54) PARENTERAL KETAMINE FORMULATIONS AND METHODS THEREOF

(71) Applicant: NRx Pharmaceuticals, Inc., Wilmington, DE (US)

(72) Inventors: Jonathan Javitt, Wilmington, DE (US); Riccardo Panicucci, Wilmington, DE (US)

(21) Appl. No.: 19/197,723

(22) Filed: May 2, 2025

(51) Int. Cl. A61K 31/135; A61J 1/05; A61K 9/08; A61K 47/02

(52) U.S. Cl. CPC A61K 31/135; A61J 1/05; A61K 9/08; A61K 47/02

(57) ABSTRACT

Disclosed in certain embodiments is a pharmaceutical composition for parenteral administration comprising: an aqueous solution comprising ketamine or a pharmaceutically acceptable salt thereof; and a pharmaceutically acceptable excipient; wherein the formulation does not comprise a preservative.

### FIELD OF THE INVENTION

In certain embodiments, the present invention relates to preservative-free parenteral ketamine formulations, method of treatment thereof and methods of preparation thereof.

### BACKGROUND OF THE INVENTION

Ketamine is a cyclohexanone derivative and rapidly acts and produces anesthesia and analgesia. Its chemical name is when in the hydrochloride salt form is ±)-2-(o-chlorophenyl)-2-(methylamino)cyclohexanone hydrochloride. Ketamine is a racemic mixture of both (R)- and (S)-enantiomers, whereas esketamine consists solely of the (S)-enantiomer. Ketamine is available as a parenteral formulation whereas esketamine is available as an intranasal formulation.

The mechanism of action of ketamine is as a noncompetitive N-methyl-D-aspartate (NMDA) and glutamate receptor antagonist that blocks HCN1 receptors. The unique dissociative action and partial agonism of opiate mu-receptors produced by ketamine allows for the performance of permits painful procedures while providing a consistent state of sedation and comfort.

The agent is highly effective for brief medical procedures that do not necessitate skeletal muscle relaxation and can be utilized as a pre-anesthetic for the induction of general anesthesia when combined with other general anesthetic agents. Furthermore, ketamine is FDA-approved for enhancing the effects of low-potency substances such as nitrous oxide.

Ketamine has also been contemplated for use in treating severe pain (e.g., associated with trauma, fractures, abdominal and flank pain, low back pain, and extremity pain). In addition to its anesthetic applications, ketamine has been contemplated for pain management, addressing treatment-resistant depression, managing suicidal ideation, and treating refractory status epilepticus

As ketamine is available as a parenteral formulation, it is susceptible to the risks associated with such formulations such as contamination and stability concerns. Generally, parenteral formulations must be sterile and not contain degradants or contaminants upon manufacture or upon storage prior to being administered to patients.

There exists a need in the art for ketamine formulations that meet the stringent requirements of parenteral administration and that can be used in the treatment of various conditions that are responsive to ketamine therapy.

### OBJECTS AND SUMMARY

It is an object of certain embodiments of the present invention to provide a parenteral ketamine formulation.

It is an object of certain embodiments of the present invention to provide a parenteral ketamine formulation that is preservative free.

It is an object of certain embodiments of the present invention to provide a parenteral ketamine formulation that is storage stable.

It is an object of certain embodiments of the present invention to provide a parenteral ketamine formulation that is suitable for the treatment of various conditions such as treatment-resistant depression and the management of suicidal ideation.

It is an object of certain embodiments of the present invention to provide methods of treating diseases and/or conditions that are amenable to ketamine therapy comprising administering a pharmaceutical composition as disclosed herein.

It is an object of certain embodiments of the present invention to provide methods of preparing a pharmaceutical composition as disclosed herein.

At least one of the above objects and others, may be met by one or more embodiments of the present invention as disclosed herein.

### BRIEF DESCRIPTION OF THE DRAWINGS

FIG. 1 depicts a blow-filled, filled and sealed system of an embodiment of the present invention.

FIG. 2 depicts an exemplary process for preparing a system of the present invention.

### DETAILED DESCRIPTION

In certain embodiments, the present invention is directed to a pharmaceutical composition for parenteral administration comprising: an aqueous solution comprising ketamine or a pharmaceutically acceptable salt thereof; and a pharmaceutically acceptable excipient; wherein the formulation does not comprise a preservative. For example, the pharmaceutical composition of the present invention does not comprise a preservative selected from benzalkonium chloride, sodium bisulfite, sodium bisulfate, sodium thiosulfate, ascorbate, benzododecinium bromide, chlorobutanol, thimerosal, phenylmercuric acetate, phenylmercuric borate, phenylmercuric nitrate, parabens (e.g., methylparaben, ethylparaben and propylparaben), polyvinyl alcohol, benzyl alcohol, phenylethanol, sodium benzoate, sorbic acid and polyquaternium-1.

In certain embodiments, the composition comprises ketamine or pharmaceutically acceptable salt thereof in an amount from about 1 mg/mL to about 20 mg/mL based on ketamine free base.

In certain embodiments, the composition comprises ketamine or pharmaceutically acceptable salt thereof in an amount, e.g., from about 5 mg/mL to about 15 mg/mL, from about 8 mg/mL to about 12 mg/mL, or about 10 mg/mL, all based on ketamine free base.

In certain embodiments, the pharmaceutical composition comprises ketamine hydrochloride.

In certain embodiments, the excipient comprises a tonicity agent, e.g., sodium chloride, dextrose, sucrose, trehalose, xylitol, fructose, glycerol, sorbitol, mannitol, potassium chloride, mannose, calcium chloride, magnesium chloride or a combination thereof. In alternative embodiments, the tonicity agent comprises sodium chloride, e.g., in an amount of about 1 mg/mL to about 15 mg/mL, about 3 mg/mL to about 12 mg/mL, about 5 mg/mL to about 9 mg/mL or about 7 mg/mL.

In certain embodiments, the excipient comprises a pH adjuster, e.g., hydrochloric acid, sodium hydroxide or a combination thereof. The pH of the composition may be, e.g., from about 2.5 to about 6.5, about 3 to about 6 or about 3.5 to about 5.5.

In certain embodiments, the composition has an osmolality of about 280 mOsm/L to about 340 mOsm/L.

In certain embodiments, the composition has a volume of about 0.1 mL to about 120 mL, about 1 mL to about 20 mL, about 2 mL to about 10 mL, about 3 mL to about 7 mL or
about 5 mL. In certain embodiments, the composition is contained in a single use container that provides one dose of the active agent.

The pharmaceutical composition of claim 1, having a volume of having ketamine related compound A impurities of not more than 0.2%.

In certain embodiments, the pharmaceutical composition upon manufacture or storage as disclosed herein exhibits one or more of total impurities of not more than 0.4%; particulate matter of 10 μm or less of not more than 6000 per 5 mL; particulate matter of 25 μm or less of not more than 600 per 5 mL; and bacterial endotoxins of not more than 0.4 EU/mg.

In certain embodiments, the pharmaceutical composition maintains at least about 90%, at least about 92%, at least about 95%, at least about 98% or at least about 99% of the amount of ketamine or a pharmaceutically acceptable salt thereof after storage for at least one of 1 month, 2 months, 3 months, 6 months, 9 months, 12 months, 18 months or 24 months at 25 °C±2 °C and 40%±5% RH.

In certain embodiments, the pharmaceutical composition of maintains at least about 90%, at least about 92%, at least about 95%, at least about 98% or at least about 99% of the amount of ketamine or a pharmaceutically acceptable salt thereof after storage for at least one of 1 month, 2 months, 3 months, 6 months, 9 months, 12 months, 18 months or 24 months at 30 °C±2 °C and 65%±5% RH.

In certain embodiments, the pharmaceutical composition of maintains at least about 90%, at least about 92%, at least about 95%, at least about 98% or at least about 99% of the amount of ketamine or a pharmaceutically acceptable salt thereof after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains a pH from about 3.5 to about 5.5 or about 4.0 to about 5.0 or about 4.2 to about 4.8 after storage for at least one of 1 month, 2 months, 3 months, 6 months, 9 months, 12 months, 18 months or 24 months month at 25 °C±2 °C and 40%±5% RH.

In certain embodiments, the pharmaceutical composition maintains a pH from about 3.5 to about 5.5 or about 4.0 to about 5.0 or about 4.2 to about 4.8 after storage for at least one of 1 month, 2 months, 3 months, 6 months, 9 months, 12 months, 18 months or 24 months month at 30 °C±2 °C and 65%±5% RH.

In certain embodiments, the pharmaceutical composition maintains a pH from about 3.5 to about 5.5 or about 4.0 to about 5.0 or about 4.2 to about 4.8 after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains an osmolality of about 280 mOsm/L to about 340 mOsm/L, about 290 mOsm/L to about 330 mOsm/L or about 300 mOsm/L to about 320 mOsm/L after storage for at least one of 1 month, 2 months, 3 months, 6 months, 9 months, 12 months, 18 months or 24 months at 25 °C±2 °C and 40%±5% RH.

In certain embodiments, the pharmaceutical composition maintains an osmolality of about 280 mOsm/L to about 340 mOsm/L, about 290 mOsm/L to about 330 mOsm/L or about 300 mOsm/L to about 320 mOsm/L after storage for at least one of 1 month, 2 months, 3 months, 6 months, 9 months, 12 months, 18 months or 24 months at 30 °C±2 °C and 65%±5% RH.

In certain embodiments, the pharmaceutical composition maintains an osmolality of about 280 mOsm/L to about 340 mOsm/L, about 290 mOsm/L to about 330 mOsm/L or about 300 mOsm/L to about 320 mOsm/L after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains ketamine related compound A impurities of not more than 0.2%, not more than 0.15% or not more than about 0.1% after storage for at least one of 1 month, 2 months, 3 months, 6 months, 9 months, 12 months, 18 months or 24 months at 25 °C±2 °C and 40%±5% RH.

In certain embodiments, the pharmaceutical composition maintains ketamine related compound A impurities of not more than 0.2%, not more than 0.15% or not more than about 0.1% after storage for at least one of 1 month, 2 months, 3 months, 6 months, 9 months, 12 months, 18 months or 24 months at 30 °C±2 °C and 65%±5% RH.

In certain embodiments, the pharmaceutical composition maintains ketamine related compound A impurities of not more than 0.2%, not more than 0.15% or not more than about 0.1% after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains individual unspecified impurities of not more than 0.2%, not more than 0.15% or not more than about 0.1% after storage for at least one of 1 month, 2 months, 3 months, 6 months, 9 months, 12 months, 18 months or 24 months at 25 °C±2 °C and 40%±5% RH.

In certain embodiments, the pharmaceutical composition maintains individual unspecified impurities of not more than 0.2%, not more than 0.15% or not more than about 0.1% after storage for at least one of 1 month, 2 months, 3 months, 6 months, 9 months, 12 months, 18 months or 24 months at 30 °C±2 °C and 65%±5% RH.

In certain embodiments, the pharmaceutical composition maintains individual unspecified impurities of not more than 0.2%, not more than 0.15% or not more than about 0.1% after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains total impurities of not more than 0.40%, not more than 0.30% or not more than about 0.20% after storage for at least one of 1 month, 2 months, 3 months, 6 months, 9 months, 12 months, 18 months or 24 months at 25 °C±2 °C and 40%±5% RH.

In certain embodiments, the pharmaceutical composition maintains total impurities of not more than 0.40%, not more than 0.30% or not more than about 0.20% after storage for at least one of 1 month, 2 months, 3 months, 6 months, 9 months, 12 months, 18 months or 24 months at 30 °C±2 °C and 65%±5% RH.

In certain embodiments, the pharmaceutical composition maintains total impurities of not more than 0.40%, not more than 0.30% or not more than about 0.20% after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains particulate matter of 10 μm or less of not more than 6,000, not more than 5,000 or not more than 4,000 per 5 mL after storage for at least one of 1 month, 2 months, 3 months, 6 months, 9 months, 12 months, 18 months or 24 months at 25 °C±2 °C and 40%±5% RH.

In certain embodiments, the pharmaceutical composition maintains particulate matter of 10 μm or less of not more than 6,000, not more than 5,000 or not more than 4,000 per 5 mL after storage for at least one of 1 month, 2 months, 3 months, 6 months, 9 months, 12 months, 18 months or 24 months at 30 °C±2 °C and 65%±5% RH.

In certain embodiments, the pharmaceutical composition maintains particulate matter of 10 μm or less of not more than 6,000, not more than 5,000 or not more than 4,000 per 5 mL after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains particulate matter of 25 μm or less of not more than 600, not more than 500 or not more than 400 per 5 mL after storage for at least one of 1 month, 2 months, 3 months, 6 months, 9 months, 12 months, 18 months or 24 months at 25 °C±2 °C and 40%±5% RH.

In certain embodiments, the pharmaceutical composition maintains particulate matter of 25 μm or less of not more than 600, not more than 500 or not more than 400 per 5 mL after storage for at least one of 1 month, 2 months, 3 months, 6 months, 9 months, 12 months, 18 months or 24 months at 30 °C±2 °C and 65%±5% RH.

In certain embodiments, the pharmaceutical composition maintains particulate matter of 25 μm or less of not more than 600, not more than 500 or not more than 400 per 5 mL after storage for at least one of 1 month, 2 months, 3 months, 6 months, 9 months, 12 months, 18 months or 24 months at 40 °C±2 °C and 25%±5% RH.

In certain embodiments, the pharmaceutical composition maintains bacterial endotoxins of not more than 0.4 EU/mg, not more than 0.3 EU/mg or not more than 0.2 EU/mg after storage for at least one of 1 month, 2 months, 3 months, 6 months, 9 months, 12 months, 18 months or 24 months at 25 °C±2 °C and 40%±5% RH.

In certain embodiments, the pharmaceutical composition maintains bacterial endotoxins of not more than 0.4 EU/mg, not more than 0.3 EU/mg or not more than 0.2 EU/mg after storage for at least one of 1 month, 2 months, 3 months, 6 months, 9 months, 12 months, 18 months or 24 months at 30 °C±2 °C and 65%±5% RH.

In certain embodiments, the pharmaceutical composition maintains bacterial endotoxins of not more than 0.4 EU/mg, not more than 0.3 EU/mg or not more than 0.2 EU/mg after storage for at least one of 1 month, 2 months, 3 months, 6 months, 9 months, 12 months, 18 months or 24 months at 40 °C±2 °C and 25%±5% RH.

In certain embodiments, the present invention is directed to a method of treating a disease or condition amenable to ketamine therapy comprising parenterally administering to a patient in need thereof a pharmaceutical composition as disclosed herein.

In certain embodiments, the patient is in need of general anesthesia and/or sedation.

In certain embodiments, the patient is treated for pain, e.g., one or more of trauma, fractures, abdominal pain, flank pain, lower back pain, and extremity pain.

In certain embodiments, the patient is treated for depression, bipolar depression, treatment-resistant depression and/or to manage suicidal ideation.

In certain embodiments, the present invention is directed to the treatment of acute stabilization of suicidal ideation in patients with depression.

In certain embodiments, the present invention is directed to the treatment of refractory status epilepticus

In certain embodiments, the parenteral administration is by a route selected from one or more of intravenous, subcutaneous, intramuscular, epidural, intraspinal, intrathecal, intracerebral, intraarticular, intracardiac, intradermal, intraperitoneal, intravitreal, intraarterial, intraorbital, and transtracheal.

In certain embodiments, the parenteral administration is by intravenous infusion, e.g.,
from about 0.1 mg/kg to about 5 mg/kg; from about 0.2 mg/kg to about 2 mg/kg; from about 0.3 mg/kg to about 1 mg/kg or about 0.5 mg/kg.

In certain embodiments, the administration is an intravenous infusion, e.g., for a time of about 5 minutes to about 6 hours, about 15 minutes to about 3 hours, about 30 minutes to about 2 hours, about 45 minutes to about 75 minutes or about 1 hour.

The administration can single or can be repeated for as long as clinically needed on a schedule or pro re nata (prn) basis. In certain embodiments, the intravenous information is administered once weekly, twice weekly, thrice weekly, daily, every other day, every third, every fourth day, every fifth day, every sixth day, every ten days, every other week, every third week or once monthly.

In a particular embodiment, the administration is about 0.5 mg/kg by intravenous infusion for about an hour, once or twice weekly.

In certain embodiments, the present invention is directed to a method of preparing a pharmaceutical composition for parenteral administration as disclosed herein comprising mixing water, ketamine or a pharmaceutically acceptable salt thereof; and a pharmaceutically acceptable excipient; wherein the formulation does not comprise a preservative.

In certain embodiments, the mixing can be in any suitable container such as a formulation tank.

In certain embodiments, the mixing is at a speed of about 10 rpm to about 1,000 rpm, from about 100 rpm to about 500 rpm, from about 300 rpm to about 400 rpm or about 340 rpm to about 360 rpm.

In certain embodiments, the mixing is for a time of about 1 minute to about 10 hours, from about 10 minutes to about 5 hours, about 15 minutes to about 2 hours or about 30 minutes to about 1 hour.

In certain embodiments, the mixture is cooled to a temperature of about 10 °C to about 35 °C, or about 20 °C to about 30 °C.

In certain embodiments, wherein the mixture comprises water, sodium chloride and ketamine hydrochloride.

In certain embodiments, sodium chloride is added to the water and ketamine hydrochloride is added to the mixture after the sodium chloride. In other embodiments, additional water is added to the mixture after the ketamine hydrochloride.

In certain embodiments, the mixture reaches a weight of about 10 kg to about 5,000 kg, from about 100 kg to about 2,000 kg, from about 150 kg to about 500 kg, or about 350 kg to about 450 kg.

In certain embodiments, the present invention is directed to a method of packaging a pharmaceutical composition as disclosed herein, comprising containing the formulation in a glass or plastic container. The plastic can be, e.g., polyethylene polypropylene, or an acrylic resin. In certain embodiments, the plastic is a polypropylene resin.

In certain plastic embodiments, the packaging comprises blow-forming the container, filling the container and sealing the container (BFS).

In certain plastic embodiments, the container is formed by extrusion. The plastic may be fed into the extruder, e.g., in the form of pellets by a hopper.

In certain embodiments, the extruder has multiple zones. The extruder ban be heated to a temperature, e.g., of about 150 °C to about 250 °C, about 175 °C to about 225 °C or about 190 °C to about 200 °C.

In certain embodiments, the extrusion forms a parison. In other embodiments, the extrusion (i.e., extrudate) is extruded into vial molds, e.g., with vacuum. The molds can, e.g., form a luer lock. In certain embodiments, fill needles descend from the parison head, core the mold, fill the vials with the composition and retract. Ther mold can utilize, e.g., a rotary mold configuration.

In certain embodiments, the present invention is directed to a system comprising a composition as disclosed herein contained in a glass or plastic container as disclosed herein. The system can be prepared according to the methods disclosed herein.

As used herein, the term “about” in connection with a measured quantity (such as a period of time, a weight, a volume a percentage, etc.) refers to the normal variations in the respective measured quantity, as expected by one of ordinary skill in the art in making the measurement and exercising a level of care commensurate with the objective of measurement and the precision of the measuring equipment. If not otherwise mentioned, all values of measured or measurable quantities disclosed herein-even in cases where these are not preceded by an “about”—are meant to include the said normal variations in the respective measured quantity.

As used herein, the singular forms “a,” “an”, and “the” include plural references unless the context clearly indicates otherwise.

As used herein the term “salt” can include, but is not limited to, inorganic acid salts such as hydrochloride, hydrobromide, hydroiodite, sulfate, phosphate and the like; organic acid salts such as formate, acetate, trifluoroacetate, maleate, tartrate, glutarate and the like; sulfonates such as methanesulfonate, benzenesulfonate, p-toluenesulfonate, and the like; and metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, dicyclohexylamine salt, N,N′-dibenzylethylenediamine salt and the like.

For the purpose of the present disclosure, any ranges defined by an upper limit and a lower limit are also meant to include all individual values or ranges between these limits.

In addition to the disclosure above, the present invention may be directed to one or more of the following items.
1. A pharmaceutical composition for parenteral administration comprising:
   an aqueous solution comprising ketamine or a pharmaceutically acceptable salt thereof; and
   a pharmaceutically acceptable excipient;
   wherein the formulation does not comprise a preservative.
2. The pharmaceutical composition of item 1, wherein the composition comprises ketamine or pharmaceutically acceptable salt thereof in an amount from about 1 mg/mL to about 20 mg/mL based on ketamine free base.
3. The pharmaceutical composition of any preceding item, wherein the composition comprises ketamine or pharmaceutically acceptable salt thereof in an amount from about 5 mg/mL to about 15 mg/mL based on ketamine free base.
4. The pharmaceutical composition of any preceding item, wherein the composition comprises ketamine or pharmaceutically acceptable salt thereof in an amount from about 8 mg/mL to about 12 mg/mL based on ketamine free base.
5. The pharmaceutical composition of any preceding item, wherein the composition comprises ketamine or pharmaceutically acceptable salt thereof in an amount of about 10 mg/mL based on ketamine free base.
6. The pharmaceutical composition of any preceding item, comprising ketamine hydrochloride.
7. The pharmaceutical composition of any preceding item, wherein the excipient comprises a tonicity agent.
8. The pharmaceutical composition of item 7, which the tonicity agent comprises sodium chloride, dextrose, sucrose, trehalose, xylitol, fructose, glycerol, sorbitol, mannitol, potassium chloride, mannose, calcium chloride, magnesium chloride or a combination thereof.
9. The pharmaceutical composition of item 7, wherein the tonicity agent comprises sodium chloride.
10. The pharmaceutical composition of item 9, wherein the composition comprises sodium chloride in an amount of about 1 mg/mL to about 15 mg/mL.
11. The pharmaceutical composition of item 9, wherein the composition comprises sodium chloride in an amount of about 3 mg/mL to about 12 mg/mL.
12. The pharmaceutical composition of item 9, wherein the composition comprises sodium chloride in an amount of about 5 mg/mL to about 9 mg/mL.
13. The pharmaceutical composition of item 9, wherein the composition comprises sodium chloride in an amount of about 7 mg/mL.
14. The pharmaceutical composition of any preceding item, wherein the excipient comprises a pH adjuster.
15. The pharmaceutical composition of item 14, wherein the pH adjuster comprises hydrochloric acid, sodium hydroxide or a combination thereof.
16. The pharmaceutical composition of any preceding item, having a pH from about 2.5 to about 6.5.
17. The pharmaceutical composition of any preceding item, having a pH from about 3 to about 6.
18. The pharmaceutical composition of any preceding item, having a pH from about 3.5 to about 5.5.
19. The pharmaceutical composition of any preceding item, having an osmolality of about 280 mOsm/L to about 340 mOsm/L.
20. The pharmaceutical composition of any preceding item, having a volume of about 0.1 mL to about 120 mL.
21. The pharmaceutical composition of any preceding item, having a volume of about 1 mL to about 20 mL.
22. The pharmaceutical composition of any preceding item, having a volume of about 2 mL to about 10 mL.
23. The pharmaceutical composition of any preceding item, having a volume of about 3 mL to about 7 mL.
24. The pharmaceutical composition of any preceding item, having a volume of about 5 mL.
25. The pharmaceutical composition of any preceding item, having ketamine related compound A impurities of not more than 0.2%.
26. The pharmaceutical composition of any preceding item, having individual unspecified impurities of not more than 0.2%.
27. The pharmaceutical composition of any preceding item 1, having total impurities of not more than 0.4%
28. The pharmaceutical composition of any preceding item, having particulate matter of 10 μm or less of not more than 6,000 per 5 mL.
29. The pharmaceutical composition of any preceding item, having particulate matter of 25 μm or less of not more than 600 per 5 mL.
30. The pharmaceutical composition of any preceding item, having bacterial endotoxins of not more than 0.4 EU/mg.
31. The pharmaceutical composition of any preceding item, which maintains at least 90% of the amount of ketamine or a pharmaceutically acceptable salt thereof after storage for 1 month at 25 °C±2 °C and 40%±5% RH.
32. The pharmaceutical composition of any preceding item, which maintains at least 90% of the amount of ketamine or a pharmaceutically acceptable salt thereof after storage for 3 months at 25 °C±2 °C and 40%±5% RH.
33. The pharmaceutical composition of any preceding item, which maintains at least 90% of the amount of ketamine or a pharmaceutically acceptable salt thereof after storage for 6 months at 25 °C±2 °C and 40%±5% RH.
34. The pharmaceutical composition of any preceding item, which maintains at least 90% of the amount of ketamine or a pharmaceutically acceptable salt thereof after storage for 9 months at 25 °C±2 °C and 40%±5% RH.
35. The pharmaceutical composition of any preceding item, which maintains at least 90% of the amount of ketamine or a pharmaceutically acceptable salt thereof after storage for 12 months at 25 °C±2 °C and 40%±5% RH.
36. The pharmaceutical composition of any preceding item, which maintains at least 90% of the amount of ketamine or a pharmaceutically acceptable salt thereof after storage for 18 months at 25 °C±2 °C and 40%±5% RH.
37. The pharmaceutical composition of any preceding item, which maintains at least 90% of the amount of ketamine or a pharmaceutically acceptable salt thereof after storage for 24 months at 25 °C±2 °C and 40%±5% RH.
38. The pharmaceutical composition of any preceding item, which maintains at least 95% of the amount of ketamine or a pharmaceutically acceptable salt thereof after storage for 1 month at 25 °C±2 °C and 40%±5% RH.
39. The pharmaceutical composition of any preceding item, which maintains at least 95% of the amount of ketamine or a pharmaceutically acceptable salt thereof after storage for 3 months at 25 °C±2 °C and 40%±5% RH.
40. The pharmaceutical composition of any preceding item, which maintains at least 95% of the amount of ketamine or a pharmaceutically acceptable salt thereof after storage for 6 months at 25 °C±2 °C and 40%±5% RH.
41. The pharmaceutical composition of any preceding item, which maintains at least 95% of the amount of ketamine or a pharmaceutically acceptable salt thereof after storage for 9 months at 25 °C±2 °C and 40%±5% RH.
42. The pharmaceutical composition of any preceding item, which maintains at least 95% of the amount of ketamine or a pharmaceutically acceptable salt thereof after storage for 12 months at 25 °C±2 °C and 40%±5% RH.
43. The pharmaceutical composition of any preceding item, which maintains at least 95% of the amount of ketamine or a pharmaceutically acceptable salt thereof after storage for 18 months at 25 °C±2 °C and 40%±5% RH.
44. The pharmaceutical composition of any preceding item, which maintains at least 95% of the amount of ketamine or a pharmaceutically acceptable salt thereof after storage for 24 months at 25 °C±2 °C and 40%±5% RH.
45. The pharmaceutical composition of any preceding item, which maintains at least 90% of the amount of ketamine or a pharmaceutically acceptable salt thereof after storage for 1 month at 30 °C±2 °C and 65%±5% RH.
46. The pharmaceutical composition of any preceding item, which maintains at least 90% of the amount of ketamine or a pharmaceutically acceptable salt thereof after storage for 2 months at 30 °C±2 °C and 65%±5% RH.
47. The pharmaceutical composition of any preceding item, which maintains at least 90% of the amount of ketamine or a pharmaceutically acceptable salt thereof after storage for 3 months at 30 °C±2 °C and 65%±5% RH.
48. The pharmaceutical composition of any preceding item, which maintains at least 90% of the amount of ketamine or a pharmaceutically acceptable salt thereof after storage for 6 months at 30 °C±2 °C and 65%±5% RH.
49. The pharmaceutical composition of any preceding item, which maintains at least 90% of the amount of ketamine or a pharmaceutically acceptable salt thereof after storage for 9 months at 30 °C±2 °C and 65%±5% RH.
50. The pharmaceutical composition of any preceding item, which maintains at least 90% of the amount of ketamine or a pharmaceutically acceptable salt thereof after storage for 12 months at 30 °C±2 °C and 65%±5% RH.
51. The pharmaceutical composition of any preceding item, which maintains at least 95% of the amount of ketamine or a pharmaceutically acceptable salt thereof after storage for 1 month at 30 °C±2 °C and 65%±5% RH.
52. The pharmaceutical composition of any preceding item, which maintains at least 95% of the amount of ketamine or a pharmaceutically acceptable salt thereof after storage for 2 month at 30 °C±2 °C and 65%±5% RH.
53. The pharmaceutical composition of any preceding item, which maintains at least 95% of the amount of ketamine or a pharmaceutically acceptable salt thereof after storage for 3 months at 30 °C±2 °C and 65%±5% RH.
54. The pharmaceutical composition of any preceding item, which maintains at least 95% of the amount of ketamine or a pharmaceutically acceptable salt thereof after storage for 6 months at 30 °C±2 °C and 65%±5% RH.
55. The pharmaceutical composition of any preceding item, which maintains at least 95% of the amount of ketamine or a pharmaceutically acceptable salt thereof after storage for 9 months at 30 °C±2 °C and 65%±5% RH.
56. The pharmaceutical composition of any preceding item, which maintains at least 95% of the amount of ketamine or a pharmaceutically acceptable salt thereof after storage for 12 months at 30 °C±2 °C and 65%±5% RH.
57. The pharmaceutical composition of any preceding item, which maintains at least 90% of the amount of ketamine or a pharmaceutically acceptable salt thereof after storage for 1 months at 40 °C±2 °C and 25%±5% RH.
58. The pharmaceutical composition of any preceding item, which maintains at least 90% of the amount of ketamine or a pharmaceutically acceptable salt thereof after storage for 2 months at 40 °C±2 °C and 25%±5% RH.
59. The pharmaceutical composition of any preceding item, which maintains at least 90% of the amount of ketamine or a pharmaceutically acceptable salt thereof after storage for 3 months at 40 °C±2 °C and 25%±5% RH.
60. The pharmaceutical composition of any preceding item, which maintains at least 90% of the amount of ketamine or a pharmaceutically acceptable salt thereof after storage for 6 months at 40 °C±2 °C and 25%±5% RH.
61. The pharmaceutical composition of any preceding item, which maintains at least 95% of the amount of ketamine or a pharmaceutically acceptable salt thereof after storage for 1 month at 40 °C±2 °C and 25%±5% RH.
62. The pharmaceutical composition of any preceding item, which maintains at least 95% of the amount of ketamine or a pharmaceutically acceptable salt thereof after storage for 2 month at 40 °C±2 °C and 25%±5% RH.
63. The pharmaceutical composition of any preceding item, which maintains at least 95% of the amount of ketamine or a pharmaceutically acceptable salt thereof after storage for 3 months at 40 °C±2 °C and 25%±5% RH.
64. The pharmaceutical composition of any preceding item, which maintains at least 95% of the amount of ketamine or a pharmaceutically acceptable salt thereof after storage for 6 months at 40 °C±2 °C and 25%±5% RH.
65. The pharmaceutical composition of any preceding item, which maintains a pH from about 3.5 to about 5.5 after storage for 1 month at 25 °C±2 °C and 40%±5% RH.
66. The pharmaceutical composition of any preceding item, which maintains a pH from about 3.5 to about 5.5 after storage for 3 months at 25 °C±2 °C and 40%±5% RH.
67. The pharmaceutical composition of any preceding item, which maintains a pH from about 3.5 to about 5.5 after storage for 6 months at 25 °C±2 °C and 40%±5% RH.
68. The pharmaceutical composition of any preceding item, which maintains a pH from about 3.5 to about 5.5 after storage for 9 months at 25 °C±2 °C and 40%±5% RH.
69. The pharmaceutical composition of any preceding item, which maintains a pH from about 3.5 to about 5.5 after storage for 12 months at 25 °C±2 °C and 40%±5% RH.
70. The pharmaceutical composition of any preceding item, which maintains a pH from about 3.5 to about 5.5 after storage for 18 months at 25 °C±2 °C and 40%±5% RH.
71. The pharmaceutical composition of any preceding item, which maintains a pH from about 3.5 to about 5.5 after storage for 24 months at 25 °C±2 °C and 40%±5% RH.
72. The pharmaceutical composition of any preceding item, which maintains a pH from about 3.5 to about 5.5 after storage for 1 month at 30 °C±2 °C and 65%±5% RH.
73. The pharmaceutical composition of any preceding item, which maintains a pH from about 3.5 to about 5.5 after storage for 2 months at 30 °C±2 °C and 65%±5% RH.
74. The pharmaceutical composition of any preceding item, which maintains a pH from about 3.5 to about 5.5 after storage for 3 months at 30 °C±2 °C and 65%±5% RH.
75. The pharmaceutical composition of any preceding item, which maintains a pH from about 3.5 to about 5.5 after storage for 6 months at 30 °C±2 °C and 65%±5% RH.
76. The pharmaceutical composition of any preceding item, which maintains a pH from about 3.5 to about 5.5 after storage for 9 months at 30 °C±2 °C and 65%±5% RH.
77. The pharmaceutical composition of any preceding item, which maintains a pH from about 3.5 to about 5.5 after storage for 12 months at 30 °C±2 °C and 65%±5% RH.
78. The pharmaceutical composition of any preceding item, which maintains a pH from about 3.5 to about 5.5 after storage for 1 month at 40 °C±2 °C and 25%±5% RH.
79. The pharmaceutical composition of any preceding item, which maintains a pH from about 3.5 to about 5.5 after storage for 2 months at 40 °C±2 °C and 25%±5% RH.
80. The pharmaceutical composition of any preceding item, which maintains a pH from about 3.5 to about 5.5 after storage for 3 months at 40 °C±2 °C and 25%±5% RH.
81. The pharmaceutical composition of any preceding item, which maintains a pH from about 3.5 to about 5.5 after storage for 6 months at 40 °C±2 °C and 25%±5% RH.
82. The pharmaceutical composition of any preceding item, which maintains an osmolality of about 280 mOsm/L to about 340 mOsm/L after storage for 1 month at 25 °C±2 °C and 40%±5% RH.
83. The pharmaceutical composition of any preceding item, which maintains an osmolality of about 280 mOsm/L to about 340 mOsm/L after storage for 3 months at 25 °C±2 °C and 40%±5% RH.
84. The pharmaceutical composition of any preceding item, which maintains an osmolality of about 280 mOsm/L to about 340 mOsm/L after storage for 6 months at 25 °C±2 °C and 40%±5% RH.
85. The pharmaceutical composition of any preceding item, which maintains an osmolality of about 280 mOsm/L to about 340 mOsm/L after storage for 9 months at 25 °C±2 °C and 40%±5% RH.
86. The pharmaceutical composition of any preceding item, which maintains an osmolality of about 280 mOsm/L to about 340 mOsm/L after storage for 12 months at 25 °C±2 °C and 40%±5% RH.
87. The pharmaceutical composition of any preceding item, which maintains an osmolality of about 280 mOsm/L to about 340 mOsm/L after storage for 18 months at 25 °C±2 °C and 40%±5% RH.
88. The pharmaceutical composition of any preceding item, which maintains an osmolality of about 280 mOsm/L to about 340 mOsm/L after storage for 24 months at 25 °C±2 °C and 40%±5% RH.
89. The pharmaceutical composition of any preceding item, which maintains an osmolality of about 280 mOsm/L to about 340 mOsm/L after storage for 1 month at 30 °C±2 °C and 65%±5% RH.
90. The pharmaceutical composition of any preceding item, which maintains an osmolality of about 280 mOsm/L to about 340 mOsm/L after storage for 2 months at 30 °C±2 °C and 65%±5% RH.
91. The pharmaceutical composition of any preceding item, which maintains an osmolality of about 280 mOsm/L to about 340 mOsm/L after storage for 3 months at 30 °C±2 °C and 65%±5% RH.
92. The pharmaceutical composition of any preceding item, which maintains an osmolality of about 280 mOsm/L to about 340 mOsm/L after storage for 6 months at 30 °C±2 °C and 65%±5% RH.
93. The pharmaceutical composition of any preceding item, which maintains an osmolality of about 280 mOsm/L to about 340 mOsm/L after storage for 9 months at 30 °C±2 °C and 65%±5% RH.
94. The pharmaceutical composition of any preceding item, which maintains an osmolality of about 280 mOsm/L to about 340 mOsm/L after storage for 12 months at 30 °C±2 °C and 65%±5% RH.
95. The pharmaceutical composition of any preceding item, which maintains an osmolality of about 280 mOsm/L to about 340 mOsm/L after storage for 1 month at 40 °C±2 °C and 25%±5% RH.
96. The pharmaceutical composition of any preceding item, which maintains an osmolality of about 280 mOsm/L to about 340 mOsm/L after storage for 2 months at 40 °C±2 °C and 25%±5% RH.
97. The pharmaceutical composition of any preceding item, which maintains an osmolality of about 280 mOsm/L to about 340 mOsm/L after storage for 3 months at 40 °C±2 °C and 25%±5% RH.
98. The pharmaceutical composition of any preceding item, which maintains an osmolality of about 280 mOsm/L to about 340 mOsm/L after storage for 6 months at 40 °C±2 °C and 25%±5% RH.
99. The pharmaceutical composition of any preceding item, which maintains ketamine related compound A impurities of not more than 0.2% after storage for 1 month at 25 °C±2 °C and 40%±5% RH.
100. The pharmaceutical composition of any preceding item, which maintains ketamine related compound A impurities of not more than 0.2% after storage for 3 months at 25 °C±2 °C and 40%±5% RH.
101. The pharmaceutical composition of any preceding item, which maintains ketamine related compound A impurities of not more than 0.2% after storage for 6 months at 25 °C±2 °C and 40%±5% RH.
102. The pharmaceutical composition of any preceding item, which maintains ketamine related compound A impurities of not more than 0.2% after storage for 9 months at 25 °C±2 °C and 40%±5% RH.
103. The pharmaceutical composition of any preceding item, which maintains ketamine related compound A impurities of not more than 0.2% after storage for 12 months at 25 °C±2 °C and 40%±5% RH.
104. The pharmaceutical composition of any preceding item, which maintains ketamine related compound A impurities of not more than 0.2% after storage for 18 months at 25 °C±2 °C and 40%±5% RH.
105. The pharmaceutical composition of any preceding item, which maintains ketamine related compound A impurities of not more than 0.2% after storage for 24 months at 25 °C±2 °C and 40%±5% RH.
106. The pharmaceutical composition of any preceding item, which maintains ketamine related compound A impurities of not more than 0.2% after storage for 1 month at 30 °C±2 °C and 65%±5% RH.
107. The pharmaceutical composition of any preceding item, which maintains ketamine related compound A impurities of not more than 0.2% after storage for 2 months at 30 °C±2 °C and 65%±5% RH.
108. The pharmaceutical composition of any preceding item, which maintains ketamine related compound A impurities of not more than 0.2% after storage for 3 months at 30 °C±2 °C and 65%±5% RH.
109. The pharmaceutical composition of any preceding item, which maintains ketamine related compound A impurities of not more than 0.2% after storage for 6 months at 30 °C±2 °C and 65%±5% RH.
110. The pharmaceutical composition of any preceding item, which maintains ketamine related compound A impurities of not more than 0.2% after storage for 9 months at 30 °C±2 °C and 65%±5% RH.
111. The pharmaceutical composition of any preceding item, which maintains ketamine related compound A impurities of not more than 0.2% after storage for 12 months at 30 °C±2 °C and 65%±5% RH.
112. The pharmaceutical composition of any preceding item, which maintains ketamine related compound A impurities of not more than 0.2% after storage for 1 month at 40 °C±2 °C and 25%±5% RH.
113. The pharmaceutical composition of any preceding item, which maintains ketamine related compound A impurities of not more than 0.2% after storage for 2 months at 40 °C±2 °C and 25%±5% RH.
114. The pharmaceutical composition of any preceding item, which maintains ketamine related compound A impurities of not more than 0.2% after storage for 3 months at 40 °C±2 °C and 25%±5% RH.
115. The pharmaceutical composition of any preceding item, which maintains ketamine related compound A impurities of not more than 0.2% after storage for 6 months at 40 °C±2 °C and 25%±5% RH.
116. The pharmaceutical composition of any preceding item, which maintains individual unspecified impurities of not more than 0.2% after storage for 1 month at 25 °C±2 °C and 40%±5% RH.
117. The pharmaceutical composition of any preceding item, which maintains individual unspecified impurities of not more than 0.2% after storage for 3 months at 25 °C±2 °C and 40%±5% RH.
118. The pharmaceutical composition of any preceding item, which maintains individual unspecified impurities of not more than 0.2% after storage for 6 months at 25 °C±2 °C and 40%±5% RH.
119. The pharmaceutical composition of any preceding item, which maintains individual unspecified impurities of not more than 0.2% after storage for 9 months at 25 °C±2 °C and 40%±5% RH.
120. The pharmaceutical composition of any preceding item, which maintains individual unspecified impurities of not more than 0.2% after storage for 12 months at 25 °C±2 °C and 40%±5% RH.
121. The pharmaceutical composition of any preceding item, which maintains individual unspecified impurities of not more than 0.2% after storage for 18 months at 25 °C±2 °C and 40%±5% RH.
122. The pharmaceutical composition of any preceding item, which maintains individual unspecified impurities of not more than 0.2% after storage for 24 months at 25 °C±2 °C and 40%±5% RH.
123. The pharmaceutical composition of any preceding item, which maintains individual unspecified impurities of not more than 0.2% after storage for 1 month at 30 °C±2 °C and 65%±5% RH.
124. The pharmaceutical composition of any preceding item, which maintains individual unspecified impurities of not more than 0.2% after storage for 2 months at 30 °C±2 °C and 65%±5% RH.
125. The pharmaceutical composition of any preceding item, which maintains individual unspecified impurities of not more than 0.2% after storage for 3 months at 30 °C±2 °C and 65%±5% RH.
126. The pharmaceutical composition of any preceding item, which maintains individual unspecified impurities of not more than 0.2% after storage for 6 months at 30 °C±2 °C and 65%±5% RH.
127. The pharmaceutical composition of any preceding item, which maintains individual unspecified impurities of not more than 0.2% after storage for 9 months at 30 °C±2 °C and 65%±5% RH.
128. The pharmaceutical composition of any preceding item, which maintains individual unspecified impurities of not more than 0.2% after storage for 12 months at 30 °C±2 °C and 65%±5% RH.
129. The pharmaceutical composition of any preceding item, which maintains individual unspecified impurities of not more than 0.2% after storage for 1 month at 40 °C±2 °C and 25%±5% RH.
130. The pharmaceutical composition of any preceding item, which maintains individual unspecified impurities of not more than 0.2% after storage for 2 months at 40 °C±2 °C and 25%±5% RH.
131. The pharmaceutical composition of any preceding item, which maintains individual unspecified impurities of not more than 0.2% after storage for 3 months at 40 °C±2 °C and 25%±5% RH.
132. The pharmaceutical composition of any preceding item, which maintains individual unspecified impurities of not more than 0.2% after storage for 6 months at 40 °C±2 °C and 25%±5% RH.
133. The pharmaceutical composition of any preceding item, which maintains total impurities of not more than 0.4% after storage for 1 month at 25 °C±2 °C and 40%±5% RH.
134. The pharmaceutical composition of any preceding item, which maintains total impurities of not more than 0.4% after storage for 3 months at 25 °C±2 °C and 40%±5% RH.
135. The pharmaceutical composition of any preceding item, which maintains total impurities of not more than 0.4% after storage for 6 months at 25 °C±2 °C and 40%±5% RH.
136. The pharmaceutical composition of any preceding item, which maintains total impurities of not more than 0.4% after storage for 9 months at 25 °C±2 °C and 40%±5% RH.
137. The pharmaceutical composition of any preceding item, which maintains total impurities of not more than 0.4% after storage for 12 months at 25 °C±2 °C and 40%±5% RH.
138. The pharmaceutical composition of any preceding item, which maintains total impurities of not more than 0.4% after storage for 18 months at 25 °C±2 °C and 40%±5% RH.
139. The pharmaceutical composition of any preceding item, which maintains total impurities of not more than 0.4% after storage for 24 months at 25 °C±2 °C and 40%±5% RH.
140. The pharmaceutical composition of any preceding item, which maintains total impurities of not more than 0.4% after storage for 1 month at 30 °C±2 °C and 65%±5% RH.
141. The pharmaceutical composition of any preceding item, which maintains total impurities of not more than 0.4% after storage for 2 months at 30 °C±2 °C and 65%±5% RH.
142. The pharmaceutical composition of any preceding item, which maintains total impurities of not more than 0.4% after storage for 3 months at 30 °C±2 °C and 65%±5% RH.
143. The pharmaceutical composition of any preceding item, which maintains total impurities of not more than 0.4% after storage for 6 months at 30 °C±2 °C and 65%±5% RH.
144. The pharmaceutical composition of any preceding item, which maintains total impurities of not more than 0.4% after storage for 9 months at 30 °C±2 °C and 65%±5% RH.
145. The pharmaceutical composition of any preceding item, which maintains total impurities of not more than 0.4% after storage for 12 months at 30 °C±2 °C and 65%±5% RH.
146. The pharmaceutical composition of any preceding item, which maintains total impurities of not more than 0.4% after storage for 1 month at 40 °C±2 °C and 25%±5% RH.
147. The pharmaceutical composition of any preceding item, which maintains total impurities of not more than 0.4% after storage for 2 months at 40 °C±2 °C and 25%±5% RH.
148. The pharmaceutical composition of any preceding item, which maintains total impurities of not more than 0.4% after storage for 3 months at 40 °C±2 °C and 25%±5% RH.
149. The pharmaceutical composition of any preceding item, which maintains total impurities of not more than 0.4% after storage for 6 months at 40 °C±2 °C and 25%±5% RH.
150. The pharmaceutical composition of any preceding item, which maintains particulate matter of 10 μm or less of not more than 6,000 per 5 mL after storage for 1 month at 25 °C±2 °C and 40%±5% RH.
151. The pharmaceutical composition of any preceding item, which maintains particulate matter of 10 μm or less of not more than 6,000 per 5 mL after storage for 3 months at 25 °C±2 °C and 40%±5% RH.
152. The pharmaceutical composition of any preceding item, which maintains particulate matter of 10 μm or less of not more than 6,000 per 5 mL after storage for 6 months at 25 °C±2 °C and 40%±5% RH.
153. The pharmaceutical composition of any preceding item, which maintains particulate matter of 10 μm or less of not more than 6,000 per 5 mL after storage for 9 months at 25 °C±2 °C and 40%±5% RH.
154. The pharmaceutical composition of any preceding item 1, which maintains particulate matter of 10 μm or less of not more than 6,000 per 5 mL after storage for 12 months at 25 °C±2 °C and 40%±5% RH.
155. The pharmaceutical composition of any preceding item, which maintains particulate matter of 10 μm or less of not more than 6,000 per 5 mL after storage for 18 months at 25 °C±2 °C and 40%±5% RH.
156. The pharmaceutical composition of any preceding item, which maintains particulate matter of 10 μm or less of not more than 6,000 per 5 mL after storage for 24 months at 25 °C±2 °C and 40%±5% RH.
157. The pharmaceutical composition of any preceding item, which maintains particulate matter of 10 μm or less of not more than 6,000 per 5 mL after storage for 1 month at 30 °C±2 °C and 65%±5% RH.
158. The pharmaceutical composition of any preceding item, which maintains particulate matter of 10 μm or less of not more than 6,000 per 5 mL after storage for 2 months at 30 °C±2 °C and 65%±5% RH.
159. The pharmaceutical composition of any preceding item, which maintains particulate matter of 10 μm or less of not more than 6,000 per 5 mL after storage for 3 months at 30 °C±2 °C and 65%±5% RH.
160. The pharmaceutical composition of any preceding item, which maintains particulate matter of 10 μm or less of not more than 6,000 per 5 mL after storage for 6 months at 30 °C±2 °C and 65%±5% RH.
161. The pharmaceutical composition of any preceding item, which maintains particulate matter of 10 μm or less of not more than 6,000 per 5 mL after storage for 9 months at 30 °C±2 °C and 65%±5% RH.
162. The pharmaceutical composition of any preceding item, which maintains particulate matter of 10 μm or less of not more than 6,000 per 5 mL after storage for 12 months at 30 °C±2 °C and 65%±5% RH.
163. The pharmaceutical composition of any preceding item, which maintains particulate matter of 10 μm or less of not more than 6,000 per 5 mL after storage for 1 month at 40 °C±2 °C and 25%±5% RH.
164. The pharmaceutical composition of any preceding item, which maintains particulate matter of 10 μm or less of not more than 6,000 per 5 mL after storage for 2 months at 40 °C±2 °C and 25%±5% RH.
165. The pharmaceutical composition of any preceding item, which maintains particulate matter of 10 μm or less of not more than 6,000 per 5 mL after storage for 3 months at 40 °C±2 °C and 25%±5% RH.
166. The pharmaceutical composition of any preceding item, which maintains particulate matter of 10 μm or less of not more than 6,000 per 5 mL after storage for 6 months at 40 °C±2 °C and 25%±5% RH.
167. The pharmaceutical composition of any preceding item, which maintains particulate matter of 25 μm or less of not more than 600 per 5 mL after storage for 1 month at 25 °C±2 °C and 40%±5% RH.
168. The pharmaceutical composition of any preceding item, which maintains particulate matter of 25 μm or less of not more than 600 per 5 mL after storage for 3 months at 25 °C±2 °C and 40%±5% RH.
169. The pharmaceutical composition of any preceding item, which maintains particulate matter of 25 μm or less of not more than 600 per 5 mL after storage for 6 months at 25 °C±2 °C and 40%±5% RH.
170. The pharmaceutical composition of any preceding item, which maintains particulate matter of 25 μm or less of not more than 600 per 5 mL after storage for 9 months at 25 °C±2 °C and 40%±5% RH.
171. The pharmaceutical composition of any preceding item, which maintains particulate matter of 25 μm or less of not more than 600 per 5 mL after storage for 12 months at 25 °C±2 °C and 40%±5% RH.
172. The pharmaceutical composition of any preceding item, which maintains particulate matter of 25 μm or less of not more than 600 per 5 mL after storage for 18 months at 25 °C±2 °C and 40%±5% RH.
173. The pharmaceutical composition of any preceding item, which maintains particulate matter of 25 μm or less of not more than 600 per 5 mL after storage for 24 months at 25 °C±2 °C and 40%±5% RH.
174. The pharmaceutical composition of any preceding item, which maintains particulate matter of 25 μm or less of not more than 600 per 5 mL after storage for 1 month at 30 °C±2 °C and 65%±5% RH.
175. The pharmaceutical composition of any preceding item, which maintains particulate matter of 25 μm or less of not more than 600 per 5 mL after storage for 2 months at 30 °C±2 °C and 65%±5% RH.
176. The pharmaceutical composition of any preceding item, which maintains particulate matter of 25 μm or less of not more than 600 per 5 mL after storage for 3 months at 30 °C±2 °C and 65%±5% RH.
177. The pharmaceutical composition of any preceding item, which maintains particulate matter of 25 μm or less of not more than 600 per 5 mL after storage for 6 months at 30 °C±2 °C and 65%±5% RH.
178. The pharmaceutical composition of any preceding item, which maintains particulate matter of 25 μm or less of not more than 600 per 5 mL after storage for 9 months at 30 °C±2 °C and 65%±5% RH.
179. The pharmaceutical composition of any preceding item, which maintains particulate matter of 25 μm or less of not more than 600 per 5 mL after storage for 12 months at 30 °C±2 °C and 65%±5% RH.
180. The pharmaceutical composition of any preceding item, which maintains particulate matter of 25 μm or less of not more than 600 per 5 mL after storage for 1 month at 40 °C±2 °C and 25%±5% RH.
181. The pharmaceutical composition of any preceding item, which maintains particulate matter of 25 μm or less of not more than 600 per 5 mL after storage for 2 months at 40 °C±2 °C and 25%±5% RH.
182. The pharmaceutical composition of any preceding item, which maintains particulate matter of 25 μm or less of not more than 600 per 5 mL after storage for 3 months at 40 °C±2 °C and 25%±5% RH.
183. The pharmaceutical composition of any preceding item, which maintains particulate matter of 25 μm or less of not more than 600 per 5 mL after storage for 6 months at 40 °C±2 °C and 25%±5% RH.
184. The pharmaceutical composition of any preceding item, which maintains bacterial endotoxins of not more than 0.4 EU/mg after storage for 1 month at 25 °C±2 °C and 40%±5% RH.
185. The pharmaceutical composition of any preceding item, which maintains bacterial endotoxins of not more than 0.4 EU/mg after storage for 3 months at 25 °C±2 °C and 40%±5% RH.
186. The pharmaceutical composition of any preceding item, which maintains bacterial endotoxins of not more than 0.4 EU/mg after storage for 6 months at 25 °C±2 °C and 40%±5% RH.
187. The pharmaceutical composition of any preceding item, which maintains bacterial endotoxins of not more than 0.4 EU/mg after storage for 9 months at 25 °C±2 °C and 40%±5% RH.
188. The pharmaceutical composition of any preceding item, which maintains bacterial endotoxins of not more than 0.4 EU/mg after storage for 12 months at 25 °C±2 °C and 40%±5% RH.
189. The pharmaceutical composition of any preceding item, which maintains bacterial endotoxins of not more than 0.4 EU/mg after storage for 18 months at 25 °C±2 °C and 40%±5% RH.
190. The pharmaceutical composition of any preceding item, which maintains bacterial endotoxins of not more than 0.4 EU/mg after storage for 24 months at 25 °C±2 °C and 40%±5% RH.
191. The pharmaceutical composition of any preceding item, which maintains bacterial endotoxins of not more than 0.4 EU/mg after storage for 1 month at 30 °C±2 °C and 65%±5% RH.
192. The pharmaceutical composition of any preceding item, which maintains bacterial endotoxins of not more than 0.4 EU/mg after storage for 2 months at 30 °C±2 °C and 65%±5% RH.
193. The pharmaceutical composition of any preceding item, which maintains bacterial endotoxins of not more than 0.4 EU/mg after storage for 3 months at 30 °C±2 °C and 65%±5% RH.
194. The pharmaceutical composition of any preceding item, which maintains bacterial endotoxins of not more than 0.4 EU/mg after storage for 6 months at 30 °C±2 °C and 65%±5% RH.
195. The pharmaceutical composition of any preceding item, which maintains bacterial endotoxins of not more than 0.4 EU/mg after storage for 9 months at 30 °C±2 °C and 65%±5% RH.
196. The pharmaceutical composition of any preceding item, which maintains bacterial endotoxins of not more than 0.4 EU/mg after storage for 12 months at 30 °C±2 °C and 65%±5% RH.
197. The pharmaceutical composition of any preceding item, which maintains bacterial endotoxins of not more than 0.4 EU/mg after storage for 1 month at 40 °C±2 °C and 25%±5% RH.
198. The pharmaceutical composition of any preceding item, which maintains bacterial endotoxins of not more than 0.4 EU/mg after storage for 2 months at 40 °C±2 °C and 25%±5% RH.
199. The pharmaceutical composition of any preceding item, which maintains bacterial endotoxins of not more than 0.4 EU/mg after storage for 3 months at 40 °C±2 °C and 25%±5% RH.
200. The pharmaceutical composition of any preceding item, which maintains bacterial endotoxins of not more than 0.4 EU/mg after storage for 6 months at 40 °C±2 °C and 25%±5% RH.
201. A method of treating a disease or condition amenable to ketamine therapy comprising parenterally administering to a patient (e.g., a human patient) in need thereof a pharmaceutical composition of any of any preceding items 1-200.
202. The method of item 201, wherein the patient is in need of general anesthesia.
203. The method of item 201 or 202, wherein the patient is in need of sedation.
204. The method of any of items 201-203, wherein the patient is treated for pain.
205. The method of item 204, wherein the pain is associated with one or more of trauma, fractures, abdominal pain, flank pain, lower back pain, and extremity pain.
206. The method of any of items 201-205, wherein the patient is treated for depression.
207. The method of item 206, wherein the patient is treated for treatment-resistant depression.
208. The method of any of items 201-207, wherein the patient is used to manage suicidal ideation.
209. The method of any of items 201-208, wherein the patient treated for refractory status epilepticus.
210. The method of any of items 201-209, wherein the patient is in need of acute stabilization of suicidal ideation in patients with depression.
211. The method of item 206 or 210, wherein the depression is bipolar depression.
212. The method of any of items 201-211, wherein the parenteral administration is by a route selected from one or more of intravenous, subcutaneous, intramuscular, epidural, intraspinal, intrathecal, intracerebral, intraarticular, intracardiac, intradermal, intraperitoneal, intravitreal, intraarterial, intraorbital, and transtracheal.
213. The method of any of items 201 to 212, wherein the parenteral administration is by intravenous infusion.
214. The method of any of items 201-213, wherein the administration is from about 0.1 mg/kg to about 5 mg/kg.
215. The method of any of items 201-214, wherein the administration is from about 0.2 mg/kg to about 2 mg/kg.
216. The method of any of items 201-215, wherein the administration is from about 0.3 mg/kg to about 1 mg/kg.
217. The method of any of items 201-216, wherein the administration is about 0.5 mg/kg.
218. The method of any of items 214 to 217, wherein the administration is an intravenous infusion.
219. The method of item 213 or 218, wherein the intravenous infusion is for a time of about 5 minutes to about 6 hours, about 15 minutes to about 3 hours, about 30 minutes to about 2 hours, about 45 minutes to about 75 minutes or about 1 hour.
220. The method of claim 213, 218 or 219, wherein the intravenous infusion is single administration or once weekly, twice weekly, thrice weekly, daily, every other day, every third, every fourth day, every fifth day, every sixth day, every ten days, every other week, every third week or once monthly.
221. A method of preparing a pharmaceutical composition for parenteral administration comprising mixing water, ketamine or a pharmaceutically acceptable salt thereof; and a pharmaceutically acceptable excipient; wherein the formulation does not comprise a preservative.
222. The method of item 221, wherein the pharmaceutical composition is in accordance with any of preceding items 1-200.
223. The method of item 221 or 222, wherein the mixing is in a container such as a formulation tank.
224. The method of any of items 221-223, wherein the mixing is at a speed of about 10 rpm to about 1000 rpm, from about 100 rpm to about 500 rpm, from about 300 rpm to about 400 rpm or about 340 rpm to about 360 rpm.
225. The method of any of items 221-224, wherein the mixing is for a time of about 1 minute to about 10 hours, from about 10 minutes to about 5 hours, about 15 minutes to about 2 hours or about 30 minutes to about 1 hour.
226. The method of any of items 221-225, wherein the mixture is cooled to a temperature of about 10 °C to about 35 °C, or about 20 °C to about 30 °C.
227. The method of any of items 221-226, wherein the mixture comprises water, sodium chloride and ketamine hydrochloride.
228. The method of item 227, wherein sodium chloride is added to the water and ketamine hydrochloride is added to the mixture after the sodium chloride.
229. The method of item 228, wherein additional water is added to the mixture after the ketamine hydrochloride.
230. The method of any of items 221-229, wherein the mixture reaches a weight of about 10 kg to about 5,000 kg, from about 100 kg to about 2,000 kg, from about 150 kg to about 500 kg, or about 350 kg to about 450 kg.
231. A method of packaging a pharmaceutical composition of any of items 1-200, comprising containing the formulation is a glass or plastic container.
232. The method of item 231, wherein the container comprises polypropylene resin.
233. The method of item 231 or 232, comprising blow-forming the container, filling the container and sealing the container.
234. The method of any of items 231-233, wherein the container is formed by extrusion.
235. The method of any of items 231-234, wherein plastic is fed into an extruder by a hopper.
236. The method of items 234 or 235, wherein the extruder has multiple zones.
237. The method of items 234-236, wherein the extruder is heated to a temperature of about 150 °C to about 250 °C, about 175 °C to about 225 °C or about 190 °C to about 200 °C.
238. The method of any of items 234-237, wherein the extrusion forms a parison.
239. The method of any of items 234-238, wherein the extrusion is extruded into vial molds.
240. The method of any of items 234-239, wherein the extrusion is formed into the molds via vacuum.
241. The method of any 239 or 240, wherein the molds form a luer lock.
242. The method of any of items 238-241, wherein fill needles descend from the parison head, core the mold, fill the vials with the composition and retract.
243. The method of any of items 239-242, utilizing a rotary mold configuration.
244. A system comprising a formulation of any of any preceding items 1-200 contained in a glass or plastic container.
245. The system of any preceding item 244, wherein the container comprises polypropylene resin.
246. The system of any of items 244 or 245, wherein the container is blow-formed, filled and sealed.
247. The system of any of items 244-246, wherein the container is formed by extrusion.
248. The system of any of items 244-247, wherein plastic is fed into an extruder by a hopper.
249. The system of any of items 247-248, wherein the extruder has multiple zones.
250. The system of any of items 247-249, wherein the extruder is heated to a temperature of about 150 °C to about 250 °C, about 175 °C to about 225 °C or about 190 °C to about 200 °C.
251. The system of any of items 247-250, wherein the extrusion forms a parison.
252. The system of any of items 247-251, wherein the extrusion is extruded into vial molds.
253. The system of any of items 247-252, wherein the extrusion is formed into the molds via vacuum.
254. The system of item 252 or 253, wherein the molds form a luer lock.
255. The system of any of items 251-254, wherein fill needles descend from the parison head, core the mold, fill the vials with the composition and retract.
256. The system of any of items 252-255, utilizing a rotary mold configuration.

The following examples are included to demonstrate certain aspects and embodiments of the invention as disclosed herein. However, those skilled in the art will appreciate that the following description is illustrative only and should not in any way be taken as a restriction of the invention.

### EXAMPLES

The qualitative and quantitative composition of a parenteral ketamine hydrochloride for intravenous infusion, 50 mg/5 mL is provided in Table 1.

TABLE 1
Composition of NRX-100 (Ketamine HCl) for Injection,
for Intravenous Infusion, 50 mg/5 mL

| | | | Concentration | | |
|---|---|---|---|---|---|
| | | | | Quantity per | |
| | Quality | Pharmaceutical | Quantity | vial (mg/vial; 5 | Quantity |
| Component | Standard | Function | % (w/v) | mL fill) | (mg/mL) |
| Ketamine | USP | Active | 1.15 | 57.65 | 11.53 |
| Hydrochloride | | Pharmaceutical | | | (10.0 mg/mL |
| | | Ingredient | | | on the |
| | | | | | Ketamine |
| | | | | | basis) |
| Sodium Chloride | USP | Tonicity Agent | 0.70 | 35.00 | 7.00 |

| | | | |
|---|---|---|---|
| Water for Injection | USP | Diluent | q.s. to target volume |
| Nitrogen¹ | NF | Processing Agent | q.s. for processing |

NF = National Formulary; q.s. = quantity sufficient; USP = United States Pharmacopeia

### Formulation

Water for injection (WFI), USP, is added to the formulation tank until the tank reaches a weight of 240±20 kg. During the chilling process, the mixer is set to 350±5 rpm and the contents of the tank are cooled utilizing chilled water in the tank jacket until a temperature probe reads 20-30 °C. For each material, the appropriate label is confirmed prior to use in formulation. Raw materials are then added in the following order: Sodium Chloride (USP) and Ketamine Hydrochloride (USP). Additional WFI, USP is added to the solution until the formulation tank weight reaches the Q.S. formulation tank weight of 400±1 kg. The solution then undergoes a final mix at 350±5 rpm for 30-60 minutes until a temperature of 20-30 °C is achieved. A calibrated pH meter is used to record the pH of the bulk formulation. The tank is then pressurized with Nitrogen, NF in preparation for filling and the formulation.

### Drug Product Filling

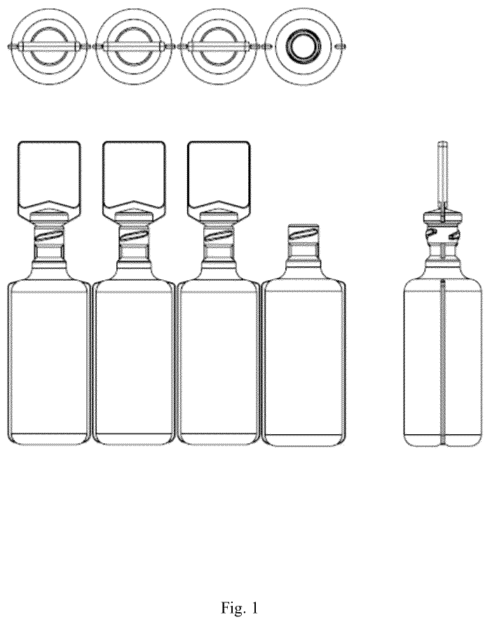

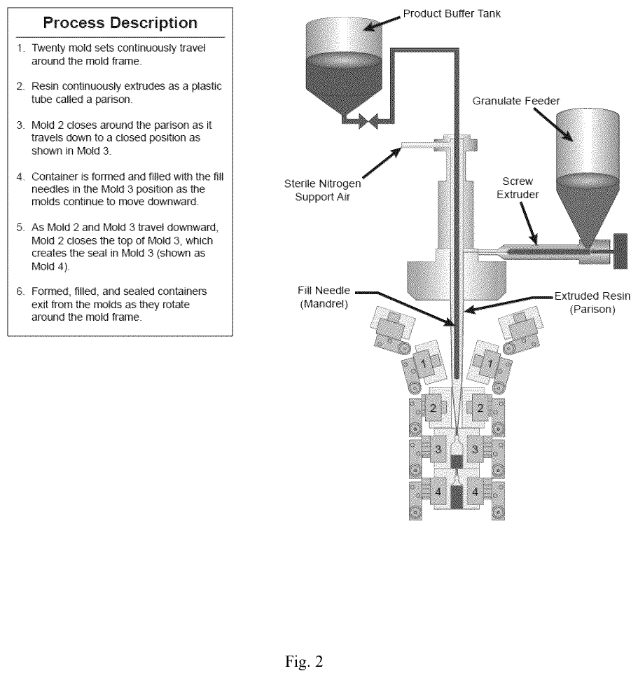

Polypropylene resin is used to manufacture the primary container closure for the drug product. The resin is virgin material, supplied in pellet form. The self-regulating hopper controls pellet demand based upon extruder requirements. Gravity propels the resin pellets through a feed from the hopper at the top of the BFS machine into the extruder. The extruder gradually heats the resin pellets in several zones from 190 to 200 °C, melting the plastic granulate to produce a sterile, extruded hollow plastic tube known as a parison. The molten polypropylene resin is extruded into the cooled vial molds and formed into the sides of the molds via vacuum. The fill needles descend from the parison head, core the luer lock, fill the vials, and retract. Next, the mold set closes, sealing the previous vials. The molds release the filled and sealed vials several cycles later due to the rotary mold configuration. The finished filled molds are shown in FIG. 1. The blow-form, fill, seal (BFS) filling process is shown in FIG. 2. Filled and sealed vials of finished drug product exit the filling suite on an automated conveyor.

### Stability Testing

Stability testing was conducted on three (3) exhibit 400 kg batches (Ex. A, Ex. B and Ex. C) of ketamine product with the following parameters in both inverted and vertical orientations:
Semi-permeable Accelerated (40 °C±2 °C, NMT 25% RH)
Semi-permeable Room Temperature (25 °C±2 °C, 40%±5% RH)
Intermediate (30 °C±2 °C, 65%±5% RH)

Table 6 summarizes the schedules for testing each exhibit batch and sets forth the storage condition and the time points for testing based on the date stability started. All three (3) exhibit lots will be stored in the semi-permeable room temperature storage condition (25 °C±2 °C, 40%±5% RH) through 730 days from induction.

| | Storage Condition | |
|---|---|---|
| Batch | (Inverted/Vertical) | Time Points (Months) |
| Ex. A | 25 °C ± 2 °C / | 1 M, 3 M, 6 M, 9 M, 12 M, 18 M, 24 M |
| | 40% ± 5% RH | |
| | 30 °C ± 2 °C / | 1 M, 2 M, 3 M, 6 M, 9 M, 12 M |
| | 65% ± 5% RH | |
| | 40 °C ± 2 °C / | 1 M, 2 M, 3 M, 6 M |
| | NMT 25% RH | |
| Ex. B | 25 °C ± 2 °C / 40% ± | 1 M, 3 M, 6 M, 9 M, 12 M, 18 M, 24 M |
| | 5% RH | |
| | 30 °C ± 2 °C / 65% ± | 1 M, 2 M, 3 M, 6 M, 9 M, 12 M |
| | 5% RH | |
| | 40 °C ± 2 °C / NMT | 1 M, 2 M, 3 M, 6 M |
| | 25% RH | |
| Ex. C | 25 °C ± 2 °C / 40% ± | 1 M, 3 M, 6 M, 9 M, 12 M, 18 M, 24 M |
| | 5% RH | |
| | 30 °C ± 2 °C / 65% ± | 1 M, 2 M, 3 M, 6 M, 9 M, 12 M |
| | 5% RH | |
| | 40 °C ± 2 °C / NMT | 1 M, 2 M, 3 M, 6 M |
| | 25% RH | |

180 day supporting data for Ex. A is presented below:

| | | |
|---|---|---|
| Test | Criteria | Result |

Ex. A 25 °C ± 2 °C/40% ± 5% RH inverted

| | | |
|---|---|---|
| pH | 3.5-5.5 | 4.5 |
| Assay | 95% to 105% | 100.8% |
| Osmolality | 280-340 mOsm/L | 309 mOsm/L |
| Ketamine Related CMP. A* | NMT 0.2% | ND |
| Individual unspecified imp. | NMT 0.2% | ND |
| Total impurities | NMT 0.4% | 0.0% |
| Visible particulates | Visual inspection | Conforms |
| Sterility | Absence of microorganisms | Conforms |
| Particulate matter ≥10 μm | NMT 6000/container | 5/container |
| Particulate matter ≥25 μm | NMY 600/container | 1/container |

Ex. A 40 °C ± 2 °C/NMT 25% RH inverted

| | | |
|---|---|---|
| pH | 3.5-5.5 | 4.4 |
| Assay | 95% to 105% | 103.8% |
| Osmolality | 280-340 mOsm/L | 317 mOsm/L |
| Ketamine Related CMP. A | NMT 0.2% | ND |
| Individual unspecified imp. | NMT 0.2% | ND |
| Total impurities | NMT 0.4% | 0.0% |
| Visible particulates | Visual inspection | Conforms |
| Sterility | Absence of microorganisms | Conforms |
| Particulate matter ≥10 μm | NMT 6000/container | 11/container |
| Particulate matter ≥25 μm | NMY 600/container | 1/container |

Ex. A 30 °C ± 2 °C/65% ± 5% RH inverted

| | | |
|---|---|---|
| pH | 3.5-5.5 | 4.5 |
| Assay | 95% to 105% | 100.9% |
| Osmolality | 280-340 mOsm/L | 307 mOsm/L |
| Ketamine Related CMP. A | NMT 0.2% | ND |
| Individual unspecified imp. | NMT 0.2% | ND |
| Total impurities | NMT 0.4% | 0.0% |
| Visible particulates | Visual inspection | Conforms |
| Sterility | Absence of microorganisms | Conforms |
| Particulate matter ≥10 μm | NMT 6000/container | 6/container |
| Particulate matter ≥25 μm | NMY 600/container | 0/container |

Ex. A 25 °C ± 2 °C/40% ± 5% RH vertical

| | | |
|---|---|---|
| pH | 3.5-5.5 | 4.5 |
| Assay | 95% to 105% | 100.8% |
| Osmolality | 280-340 mOsm/L | 307 mOsm/L |
| Ketamine Related CMP. A | NMT 0.2% | ND |
| Individual unspecified imp. | NMT 0.2% | ND |
| Total impurities | NMT 0.4% | 0.0% |
| Visible particulates | Visual inspection | Conforms |
| Sterility | Absence of microorganisms | Conforms |
| Particulate matter ≥10 μm | NMT 6000/container | 3/container |
| Particulate matter ≥25 μm | NMY 600/container | 0/container |

Ex. A 40 °C ± 2 °C/NMT 25% RH vertical

| | | |
|---|---|---|
| pH | 3.5-5.5 | 4.4 |
| Assay | 95% to 105% | 103.7% |
| Osmolality | 280-340 mOsm/L | 317 mOsm/L |
| Ketamine Related CMP. A | NMT 0.2% | ND |
| Individual unspecified imp. | NMT 0.2% | ND |
| Total impurities | NMT 0.4% | 0.0% |
| Visible particulates | Visual inspection | Conforms |
| Sterility | Absence of microorganisms | Conforms |
| Particulate matter ≥10 μm | NMT 6000/container | 6/container |
| Particulate matter ≥25 μm | NMY 600/container | 1/container |

Ex. A 30 °C ± 2 °C/65% ± 5% RH vertical

| | | |
|---|---|---|
| pH | 3.5-5.5 | 4.5 |
| Assay | 95% to 105% | 100.9% |
| Osmolality | 280-340 mOsm/L | 308 mOsm/L |
| Ketamine Related CMP. A | NMT 0.2% | ND |
| Individual unspecified imp. | NMT 0.2% | ND |
| Total impurities | NMT 0.4% | 0.0% |
| Visible particulates | Visual inspection | Conforms |
| Sterility | Absence of microorganisms | Conforms |
| Particulate matter ≥10 μm | NMT 6000/container | 1/container |
| Particulate matter ≥25 μm | NMY 600/container | 0/container |

*1-[(2-Chlorophenyl) (methylimino)methyl] cyclopentanol

1-256. (canceled)

257. A method of treating depression comprising parenterally administering to a patient in need thereof a pharmaceutical composition comprising:
an aqueous solution comprising ketamine or a pharmaceutically acceptable salt thereof;
a pharmaceutically acceptable excipient comprising sodium chloride in an amount of 5 mg/mL to 7 mg/mL and wherein the composition is adjusted to a pH from about 3.5 to about 5.5;
wherein the composition does not comprise a preservative and maintains at least 90% of the amount of ketamine or a pharmaceutically acceptable salt thereof after storage for 6 months at 25 °C±2 °C and 40%±5% RH;
wherein the composition maintains an osmolality of about 280 mOsm/L to about 340 mOsm/L after storage for 6 months at 25 °C±2 °C and 40%±5% RH;
wherein the ketamine or pharmaceutically acceptable salt thereof is the sole active agent in the composition.

258. The method of claim 257, wherein the patient is in need of acute stabilization of suicidal ideation in patients with depression.

259. The method of claim 258, wherein the parenteral administration is by intravenous infusion at about 0.3 mg/kg to about 1 mg/kg.

260. The method of claim 259, wherein the intravenous infusion is for a time of about 45 minutes to about 75 minutes.

261. The method of claim 260, wherein the intravenous infusion is single administration or twice weekly.

262. The method of claim 261, wherein the composition comprises ketamine or pharmaceutically acceptable salt thereof in an amount from about 8 mg/mL to about 12 mg/mL based on ketamine free base.

263. (canceled)

264. The method of claim 262, wherein the composition comprises ketamine hydrochloride.

265. The method of claim 264, wherein the composition maintains a pH from about 3.5 to about 5.5 after storage for 1 month at 25 °C±2 °C and 40%±5% RH.

266. (canceled)

267. The method of claim 266, wherein the composition maintains ketamine related compound A impurities of not more than 0.2% after storage for 1 month at 25 °C±2 °C and 40%±5% RH.

268. The method of claim 267, wherein the composition maintains individual unspecified impurities of not more than 0.2% after storage for 1 month at 25 °C±2 °C and 40%±5% RH.

269. The method of claim 268, wherein the composition maintains total impurities of not more than 0.4% after storage for 1 month at 25 °C±2 °C and 40%±5% RH.

270. The method of claim 269, wherein the composition maintains particulate matter of 10 μm or less of not more than 6,000 per 5 mL after storage for 1 month at 25 °C±2 °C and 40%±5% RH.

271. The method of claim 270, wherein the composition maintains particulate matter of 25 μm or less of not more than 600 per 5 mL after storage for 1 month at 25 °C±2 °C and 40%±5% RH.

272. The method of claim 271, wherein the composition maintains bacterial endotoxins of not more than 0.4 EU/mg after storage for 1 month at 25 °C±2 °C and 40%±5% RH.

273. The method of claim 272, wherein the composition is contained in a plastic container prior to administration.

274. The method of claim 273, wherein the container comprises polyolefin resin.

275. (canceled)

276. (canceled)

277. A method of treating depression comprising parenterally administering to a patient in need thereof a pharmaceutical composition comprising:
an aqueous solution comprising ketamine hydrochloride; and
a pharmaceutically acceptable excipient comprising sodium chloride in an amount of 5 mg/mL to 7 mg/mL and wherein the composition is adjusted to a pH from about 3.5 to about 5.5;
wherein the composition does not comprise a preservative and maintains at least 90% of the amount of ketamine or a pharmaceutically acceptable salt thereof after storage for 6 months at 25 °C±2 °C and 40%±5% RH;
wherein the composition maintains an osmolality of about 280 mOsm/L to about 340 mOsm/L after storage for 6 months at 25 °C±2 °C and 40%±5% RH;
wherein the composition has total impurities of not more than 0.4%; and
wherein ketamine hydrochloride is the sole active agent in the composition.

278. A method of treating depression comprising parenterally administering to a patient in need thereof a pharmaceutical composition comprising:
an aqueous solution comprising ketamine hydrochloride in an amount from about 8 mg/mL to about 12 mg/mL based on ketamine free base; and
a pharmaceutically acceptable excipient comprising sodium chloride in an amount of 5 mg/mL to 7 mg/mL and wherein the composition is adjusted to a pH from about 3.5 to about 5.5;
wherein the composition does not comprise a preservative and maintains at least 95% of the amount of ketamine or a pharmaceutically acceptable salt thereof after storage for 12 months at 25 °C±2 °C and 40%±5% RH;
wherein the composition maintains an osmolality of about 280 mOsm/L to about 340 mOsm/L after storage for 6 months at 25 °C±2 °C and 40%±5% RH;
wherein the composition has total impurities of not more than 0.4%;
wherein the composition has particulate matter of 10 μm or less of not more than 6,000 per 5 mL;
wherein the composition has bacterial endotoxins of not more than 0.4 EU/mg;
wherein ketamine hydrochloride is the sole active agent in the composition.
